(19) United States
(12) Patent Application Publication
(10) Pub. No.: US 2022/0051780 A1
(43) Pub. Date: Feb. 17, 2022

(54) System, Mobile Application and Process for an Advanced Recovery Device

(71) Applicant: Hyper Ice, Inc., Irvine, CA (US)

(72) Inventors: Gilad Jacobs, Irvine, CA (US); John Aquadro, Irvine, CA (US); James Huether, Irvine, CA (US)

(21) Appl. No.: 17/402,129

(22) Filed: Aug. 13, 2021

Related U.S. Application Data
(60) Provisional application No. 63/066,010, filed on Aug. 14, 2020.

(51) Int. Cl. G16H 20/30; G06N 5/04

(52) U.S. Cl. CPC G16H 20/30; G06N 5/04

(57) ABSTRACT

A method for operating a compressive recovery device. The method including receiving activity data of a user, providing a recommended recovery activity based on the activity data, wherein a primary goal of the recovery activity is to facilitate physiological recovery of the user, and controlling a compressive recovery device to perform the recovery activity on the user.

### CROSS-REFERENCE TO RELATED APPLICATIONS

This application claims the benefit of priority of U.S. Provisional Application No. 63/066,010 filed on Aug. 14, 2020, which is incorporated herein by reference in its entirety.

### FIELD

The present disclosure is related to the field of exercise recovery, and, more particularly, is related the field of development of personalized recovery exercise routines based on data collected from exercise tracking systems.

### BACKGROUND

Percussive massage, which is also referred to as tapotement, is the rapid, percussive tapping, slapping and cupping of an area of the human body. Percussive massage is used to more aggressively work and strengthen deep-tissue muscles. Percussive massage increases local blood circulation and can even help tone muscle areas.

Percussive massage can be applied by electromechanical percussive massage devices (percussive applicators), which are commercially available. Such percussive applicators may include, for example, an electric motor coupled to drive a reciprocating piston within a cylinder. A variety of percussive heads may be attached to the piston to provide different percussive effects on selected areas of the body

In addition, edematous conditions, i.e., excessive accumulation of fluid in tissues, are painful conditions that can arise from a variety of causes. For example, preoperative, operative and postoperative immobilization of limbs can cause blood stasis and venous thromboembolism, a serious edematous condition. The swelling of limbs in edematous conditions can be unsightly and ultimately life threatening.

Existing systems treat edema with pressure devices that squeeze the limb, typically by means of an inflatable pressure cuff wrapped around the limb. The pressure device moves excess fluid from engorged tissues from distal portions of the limb to proximal portions, eventually to the trunk of the body where the fluids are absorbed in the circulatory system and excreted from the body. These pressure devices thus perform external, non-invasive compression therapy.

Percussive massage and compression therapy can be used to facilitate recovery from exercise activity. Current recovery activities are not properly informed of the nature of the exercise activity performed by a particular individual, nor the devices available to perform the recovery activity (e.g., a percussive massage device or compression therapy device). Better techniques for determining recovery regiments are needed.

The foregoing examples of the related art and limitations therewith are intended to be illustrative and not exclusive, and are not admitted to be “prior art.” Other limitations of the related art will become apparent to those of skill in the art upon a reading of the specification and a study of the drawings.

### SUMMARY

At least one aspect of the present disclosure is directed to a method for operating a compressive recovery device. The method includes receiving activity data of a user, providing a recommended recovery activity based on the activity data, wherein a primary goal of the recovery activity is to facilitate physiological recovery of the user, and controlling a compressive recovery device to perform the recovery activity on the user.

In one embodiment, the activity data is received from a wearable activity tracker. In some embodiments, the activity data is received from a smart fitness machine. In certain embodiments, the step of controlling the compressive recovery device comprises controlling a pressure provided by the compressive recovery device. In various embodiments, controlling the pressure provided by the compressive recovery device to perform the recovery activity includes controlling the pressure in at least one zone of a plurality of zones of the compressive recovery device.

In some embodiments, providing the recommend recovery activity based on the activity data includes providing a sequence of two or more recovery activities, at least one activity of the sequence including the compressive recovery device. In one embodiment, the method includes connecting to the compressive recovery device and receiving recovery data related to the recovery activity from the compressive recovery device. In certain embodiments, the method includes comparing the recovery data to an optimal recovery data and presenting the comparison results to the user. In various embodiments, the method includes presenting the comparison results to a third party.

In one embodiment, the third party comprises at least one of a trainer or a healthcare professional. In some embodiments, the method includes providing a recommended recovery activity comprises using an artificial intelligence technique.

Another aspect of the present disclosure is directed to a system including one or more data processing apparatus programmed to perform operations including receiving activity data of a user, providing a recommended recovery activity based on the activity data, wherein a primary goal of the recovery activity is to facilitate physiological recovery of the user, and controlling a compressive recovery device to perform the recovery activity on the user.

In one embodiment, the activity data is received from a wearable activity tracker. In some embodiments, the activity data is received from a smart fitness machine. In various embodiments, the operation of controlling the compressive recovery device comprises controlling a pressure provided by the compressive recovery device. In certain embodiments, controlling the pressure provided by the compressive recovery device to perform the recovery activity includes controlling the pressure in at least one zone of a plurality of zones of the compressive recovery device.

In some embodiments, providing the recommend recovery activity based on the activity data includes providing a sequence of two or more recovery activities, at least one activity of the sequence including the compressive recovery device. In one embodiment, the operations include connecting to the compressive recovery device and receiving recovery data related to the recovery activity from the compressive recovery device. In certain embodiments, the operations include comparing the recovery data to an optimal recovery data and presenting the comparison results to the user. In various embodiments, providing a recommended recovery activity comprises using an artificial intelligence technique.

### BRIEF DESCRIPTION OF THE DRAWINGS

The accompanying figures, which are included as part of the present specification, illustrate the presently preferred embodiments and together with the generally description given above and the detailed description of the preferred embodiments given below serve to explain and teach the principles described herein.

FIG. 1 is a block diagram illustrating a network and connected devices for recommending a recovery exercise routine.

FIG. 2 illustrates an example onboarding user interface for use with an online or mobile application for recommending recovery routines.

FIG. 3 illustrates another example onboarding user interface for use with the online or mobile application for recommending recovery routines.

FIG. 4 illustrates yet another example onboarding user interface for use with the online or mobile application for recommending recovery routines.

FIG. 5 illustrates an exercise recommendation user interface for use with the online or mobile application for recommending recovery routines.

FIG. 6 illustrates a filtering user interface for use with the online or mobile application for recommending recovery routines.

FIG. 7 illustrates a muscle group selection user interface for use with the online or mobile application for recommending recovery routines.

FIG. 8 illustrates a smart recovery device scanning user interface for use with the online or mobile application for recommending recovery routines.

FIG. 9 illustrates an exercise recommendation user interface for use with the online or mobile application for recommending recovery routines.

FIG. 10 illustrates an exercise recommendation user interface for use with the online or mobile application for recommending recovery routines.

FIG. 11 illustrates a smart recovery device user interface for use with the online or mobile application for recommending recovery routines.

FIG. 12 illustrates a messaging user interface for use with the online or mobile application for recommending recovery routines.

FIG. 13 illustrates a messaging user interface for use with the online or mobile application for recommending recovery routines.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

As used throughout this specification, the words “upper,” “lower,” “longitudinal,” “upward,” “downward,” “proximal,” “distal,” and other similar directional words are used with respect to the views being described. It should be understood that the percussive massage applicator described herein can be used in various orientations and is not limited to use in the orientations illustrated in the drawing figures.

### Percussive Massage Applicator

A portable electromechanical percussive massage applicator (“percussive massage applicator”) can be applied to different locations of body to apply percussion to the body to effect percussive treatment. The percussive massage applicator is operable with removably attachable applicator heads to vary the effect of the percussive strokes. The percussive massage applicator operates at a plurality of speeds (e.g., three speeds). In one example, the percussive massage device corresponds to one or more of the devices described in U.S. patent application Ser. No. 17/372,237, filed on Jul. 9, 2021, and entitled “SYSTEM, APPLICATION, AND PROCESS FOR EXERCISE RECOVERY,” which is incorporated herein by reference in its entirety.

### Compressive Recovery Device

As described above, edematous conditions, i.e., excessive accumulation of fluid in tissues, are painful conditions that can arise from a variety of causes. For example, preoperative, operative and postoperative immobilization of limbs can cause blood stasis and venous thromboembolism, a serious edematous condition. The swelling of limbs in edematous conditions can be unsightly and ultimately life threatening.

Existing systems treat edema with pressure devices that squeeze the limb, typically by means of an inflatable pressure cuff wrapped around the limb. The pressure device moves excess fluid from engorged tissues from distal portions of the limb to proximal portions, eventually to the trunk of the body where the fluids are absorbed in the circulatory system and excreted from the body. These pressure devices thus perform external, non-invasive compression therapy.

According to one embodiment, a compressive recovery device includes three different inflatable sleeves for the legs, arms, and hips. Each sleeve is divided into zones that can be individually controlled to apply pressure pulses (dynamic compression) to a particular area of the body for a particular time. In some examples, the compressive recovery device is a compression therapy device. In one example, the compressive recovery device corresponds to one or more of the devices described in U.S. Pat. No. 5,968,073, filed on Oct. 19, 1999, and entitled “METHODS AND APPARATUS FOR APPLYING PRESSURE,” which is incorporated herein by reference in its entirety.

### Exercise Recovery System

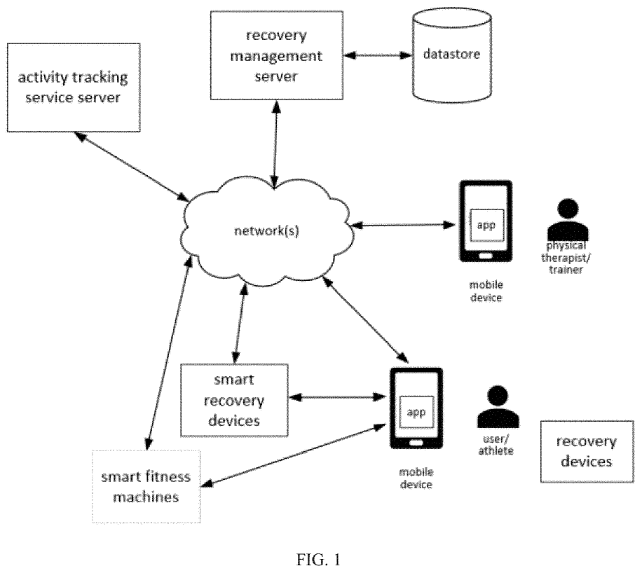

In some embodiments, the present application relates to an online or mobile based application that can be used in conjunction with a compressive recovery device or other exercise recovery device in order to facilitate improved exercise recovery. According to one embodiment, the mobile application is a recovery application for athletes and recreational users to stay injury free while they progress in training. The online or mobile application can store many recovery routines/instructions for different body parts, sports, and activities. The user can connect the application with third-party services that are used to track/record activity, e.g., Strava™, Apple Health™, Nike™, Whoop™, Garmin Connect™, Polar Flow™, Suunto App™, Peloton™, among many others. The application then makes recommendations for the best recovery routine depending on the user's training. Recovery routines may include yoga, warm-up stretching, meditation, physical therapy routines and other wellness activities. In general, the application can connect and exchange data with any known activity tracking device or service. In certain embodiments, the application can also connect directly with exercise equipment, e.g., treadmills, bikes, steppers, as an additional or alternate source of data for tracking a user's activity. The application can then provide recommendations for recovery routine(s) depending on the user's preferences, training, goals, etc. In some embodiments, the data can be shared with a third party such as a trainer, physical therapist, or medical professional. As shown in FIG. 1, the online or mobile application can be connected (e.g., via Bluetooth, WiFi, or other wireless or wired known connectivity techniques) to smart recovery devices. The smart recovery devices can include the compressive recovery devices described herein and others made available by Hyperice™ and NormaTec by Hyperice™, such as the Pulse Series recovery system, as a few examples. In various embodiments, the smart recovery devices are managed via the application according to a selected recovery routine.

The paragraphs that follow provide example descriptions of the operation of the online or mobile application according to various embodiments; however, these descriptions do not limit the broad concept described above of an application that receives activity information and provides recommended recovery activities based on a user's preferences, training, goals, etc.

### Onboarding

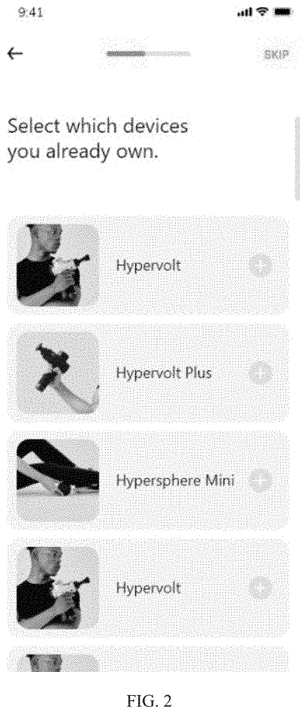

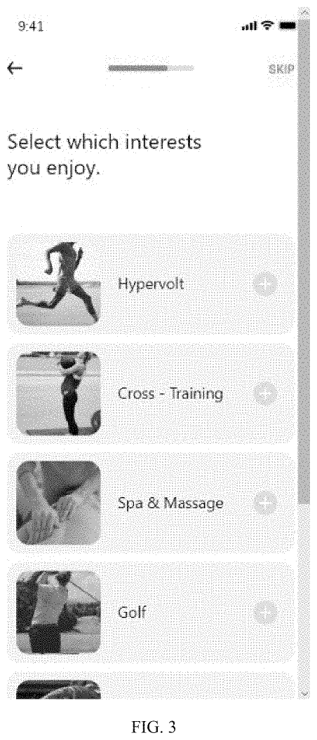

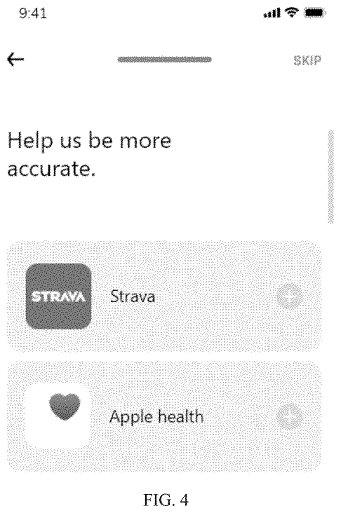

In various embodiments, a user can download the online or mobile application from known web or mobile sources, e.g., Apple's App. Store™, Google's Play Store™, etc. After the user has downloaded the application and registered, an onboarding protocol can be initiated. As shown in FIG. 2, the application can obtain information about the smart recovery devices used by the user. As shown in FIG. 3, the application can obtain information about a user's activities/interests. As shown in FIG. 4, the application can obtain information about the activity tracking devices used by the user. In general, any technique for soliciting information from the user can be used. For example, a list of selectable options can be presented, as shown in FIGS. 2-4, or a field can be presented for a user to type/insert the information. In various embodiments, additional or alternative information can also be solicited from the application during the onboarding process. As a few examples, the application can obtain user biographical information (name, sex, age, location), anatomical information (height, weight, body mass, etc.), familial information, health information, an avatar, goal information (e.g., better mobility, improved sleep, reduced soreness), etc.

Following receipt of the onboarding information, the application can provide recommendations to the user for customized activities or exercises to assist a user with accomplishing their goals. For example, the mobile application can recommend to the user customized exercises at a home screen in the category “Top picks for you” to improve a recovery routine, stay healthy, move better, etc. The recommended customized exercises can be determined based on a library of exercises accessible by the application, e.g., in a recovery management server. In addition, the recommendations may be informed using data provided from the exercise equipment or activity tracker devices used by the user. In some embodiments, the application can evaluate a user's fitness and health data to prescribe/recommend recovery routines based on any relevant factor (which in some cases can be identified by an artificial intelligence or machine learning algorithm). In some embodiments, the factors can include steps walked/run, intensity level, sleep hours, heart rate, etc. In general, recommendations can be provided using any suitable techniques. For example, the application can send notifications based on activity, sleep, heart rate, inactivity, time, etc. In some embodiments, the application can inform the user of the basis for sending a notification.

### Connecting with Third Party Services

In various instances, the user may use various third party activity tracking applications to track the user's activity. In such instances, if the user connects the online or mobile application to the activity tracking service (e.g., using known pairing techniques, such as Bluetooth), the user can be provided with customized recovery programs through the online or mobile application following at desirable times, e.g., following exercise or activity by the user, upon the user's request, etc. In certain instances, the online or mobile application described herein can be downloaded on the same hardware device (e.g., smartphone, smartwatch, etc.) as the software for the activity tracker. In other instances, activity information can be provided to the online or mobile application from an exercise machine (e.g., treadmill, bike, elliptical, stepping machine, etc.) to inform the recommended recovery program. As one example, if the user was cycling for two hours, that information can be provided to the online or mobile application either from an activity tracker or an exercise equipment. In some examples, the user can connect the online or mobile application with a desired trainer or physical/occupational therapist to achieve optimal support from during exercise and/or recovery. The online or mobile application can use that information to determine the muscles that have been used/fatigued and can suggest customized recovery exercises for these muscles (e.g., exercises, intensity levels, reps., timing, etc.) with the optimal smart recovery device and/or non-connected recovery device. In some cases, the recovery routines suggested can be informed by the smart recovery device known to be available to the user (e.g., from information provided during onboarding). In other cases, the online or mobile application can suggest recovery routines that do not require a particular device (e.g., stretching activities, etc.).

The suggested exercises can be performed with the smart recovery devices which are connected to the online or mobile application (e.g., via Bluetooth). While the online or mobile application knows the activity the user performed, and determines if the workout was low intensity or high intensity, the application can suggest an exact exercise with the optimal smart recovery device. For example, the online or mobile application can evaluate the degree of intensity based on all connected health data for a user and insights driven from aggregate user data stored in the cloud or on servers. This recovery exercise routine can be played automatically on the user's mobile device through the online or mobile application with the appropriate intensity levels for the user and for the targeted muscles associated with the activity performed by the user.

In various embodiments, the online or mobile application can also provide suggested exercise activities at other times (e.g., not immediately after a workout). In addition to stretches and exercises immediately following a workout, various maintenance exercises can also be important to improved recovery and fitness. The online or mobile application can also suggest such exercises, which can also be accessible to the application, e.g., stored in a library on a remote server or, in some cases, downloaded locally. The suggested recovery and maintenance exercises can be informed by a user's actual (e.g., daily) activity. In some cases, the online or mobile application can also provide a recommended time for performing the recovery or maintenance exercise (e.g., immediately, the day after a workout, a few days after a workout, etc.).

### Programs: Warm Up, Recovery, Maintenance

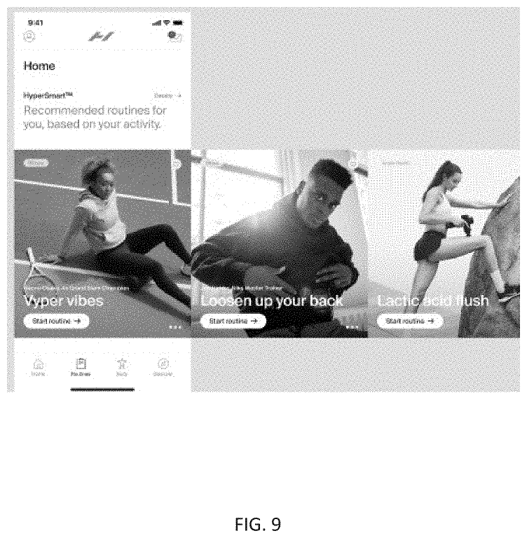

In various embodiments, the online or mobile application can present exercises to a user on a program tab or screen. For example, the exercises can be presented in a routines tab of the application. In some examples, the exercises can be presented on a home screen of the application (see FIG. 9). The exercises can be customized and tailored based on information provided by the user during onboarding and/or data received from activity tracker or exercise devices. In some instances, if the user has not answered or finished the onboarding questions, or if insufficient data is available from an activity tracker or exercise machine, the application will provide a default recovery exercise.

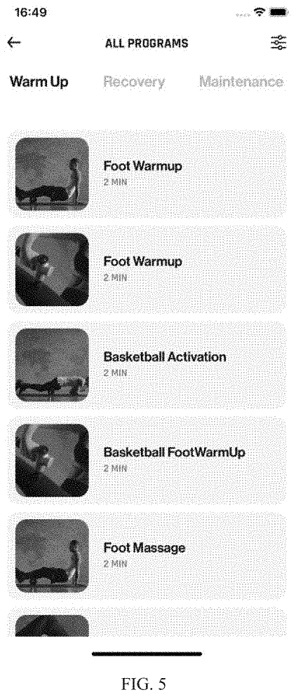

In various embodiments, the online or mobile application can sort routines based on a combination of questions answered by the user and activity data provided by activity trackers or exercise equipment. In some cases, the application can provide recommend routines based on a particular activity (e.g., “sport”) either input by the user or determined based on the received activity data. In some cases, sport-specific routines may only be provided for user's that have selected a specific sport or activity of focus. In various embodiments, the online or mobile application can also considers user ratings (e.g., ratings on a particular recovery routine), such that the recommendations are based on the feedback provided by a specific user. As shown in FIG. 5, the online or mobile application can divide the programs tab such that the exercises are presented in an organized format, e.g., recovery, warm up and maintenance exercises. In other examples, the online or mobile application can divide the programs tab such that the exercise are organized as: top rated, favorites, athletes, new, and all.

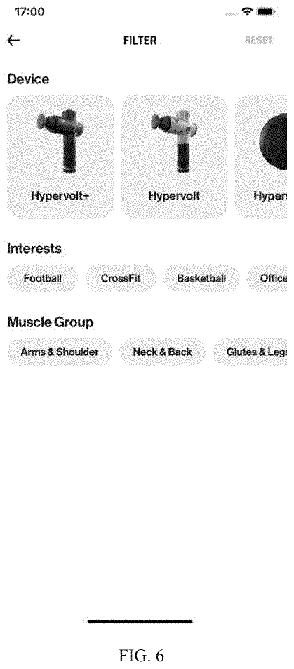

As shown in FIG. 6, for example, in various embodiments, the online or mobile application can filter the recommended (or available) exercises according to any combination of the following: (i) the available fitness machines and/or smart recovery devices registered by the user, (ii) the user's interests, and/or (iii) the targeted muscle group. The sorting/presentation features can enable users to quickly find a customized/recommended exercise routine.

In various embodiments, the online or mobile application can filter the information presented to users according to any combination of the following categories.

Features Users/Sponsored Routines: popular routines can be presented based on professional athlete routines, health advisor routines and third party partner (e.g., Equinox™, Soul Cycle™, etc.) routines. In some embodiments, videos from high profile/sponsored users can be provided to ordinary users. For example, professional athletes can provide the user with an introductory video and a series of curated routines recommend by the professional athlete.

Duration: Routines can be presented based on duration (e.g., 5 min routines, 10 minute routines, 20 minute routines, etc.).

Sport: Routines can be presented based on the applications sport (e.g., baseball, golf, yoga, etc.)

Activity Type: Routines can be presented based on activity type (e.g. endurance routines, stretching routines, sleep routines, performance routines, cool down routines, etc.)

Exercise Type: Routines can be presented based on exercise type (e.g., recovery routines, maintenance routines, etc.)

Product Type: Routines can be presented based on the product types, either recovery devices such as the compressive recovery device herein, or exercise machines (e.g., treadmill, bike, elliptical, etc.)

The online or mobile application may prescribe routines with multiple products or activities sequenced based on data metrics. As one of many examples, the present system may recommend/prescribe that the user complete an exercise using a percussive muscle recovery device first and then use a roller. The application may recommend and displays multiple product routines and determine a sequence for the routines using stored data and/or analytics (e.g., informed using artificial intelligence or machine learning).

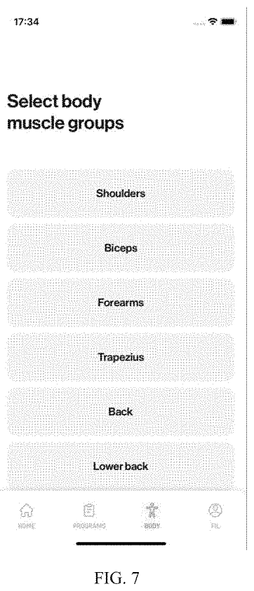

In various embodiments, in order to have quick access to certain exercises for certain muscle groups, the online or mobile application can present exercises on a “Body” tab, as shown for example in FIG. 7. Under the body tab a user can find different muscle names. If a user clicks on one muscle, he will get automatically to the screen where he can find all the exercises with that specific muscle he has chosen. On the body tab a user can also filter by a program (warm up, recovery, maintenance), by recovery devices and by interests. According to another embodiment, a human pictogram (e.g., 2D or 3D figure) allows a user to click on certain muscles on the pictogram to obtain recommended exercises connected to that body part.

In various embodiments, the online or mobile application can determine a longevity index (LI) as a standardized scale calculated from all users performing a particular activity. The LI can be determined using data stored on a remote/cloud recovery management server. In various embodiments, the LI can be based on warm up, recovery and maintenance exercises that the user performed. In some cases, a points system can be applied and assigned for various types of activities. For example, points for warm up (e.g., 2 points), recovery (e.g., 5 points) and maintenance (e.g., 3 points). In some cases, the points can be multiplied by a normalizing factor. For example, if a user did five warm up exercises, five recovery exercises and five maintenance exercises, the LI can be: (2+5+3)×5=50, according to one embodiment.

According to another embodiment, the online or mobile application (e.g., via a recovery management server) calculates an individual recovery score for the user. The recovery score is based on the user's individual performance level, activity and their warm up, recovery and maintenance exercises.

### Exercises Via Connected Devices

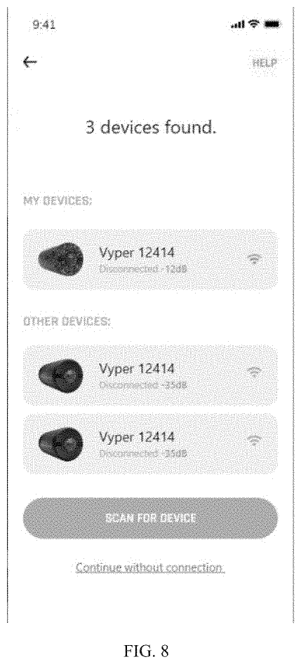

In various embodiments, exercises recommended by the application can be done with smart fitness machines and smart recovery devices (e.g., connected via Bluetooth with the mobile application). In certain embodiments, the application can recommend particular exercises and particular devices (e.g., recovery devices) to perform the exercise. In some instances, the application can allow a user to scan/search for particular exercises and/or devices, in either order (e.g., exercise first or device first). For example, after scanning, the application can present the user with all smart devices that are in the range of his mobile device. As shown in FIG. 8, in some instances, the smart device with the strongest signal (e.g., signal strength under −50 db) will appear first on his list and the user can connect with this smart device. When multiple devices are located in a scanning area, the Bluetooth scan can find the closest device to the user's mobile device by proximity. In other instances, the devices are presented in an order based on relevancy for a particular recommended exercise, as opposed to signal strength.

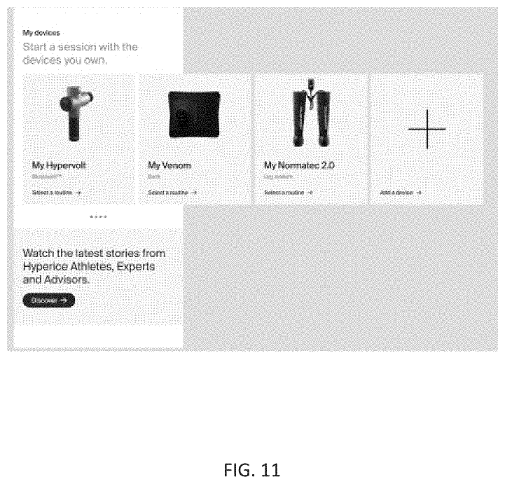

In various embodiments, prior used devices can be saved within the application (or remote server accessible thereby). For example, if a user connected to the smart device in the past, the smart device can be saved under “my device” as presented by the application (see FIG. 8 and FIG. 11). The user can choose if the exercise will be selected manually or automatically.

### Trainer/Physical Therapist Application

In various embodiments, the online or mobile application can also have a separate presentation or delivery mode for use with a personal trainer, physical therapist, or other exercise/health related professional (hereinafter referred to as “trainer application”). The trainer application can be displayed on a web-based or mobile device of the trainer (see, e.g., FIG. 1). Th trainer application can help trainers (or other exercise support professionals) support users. The trainer and the user will be connected through the recovery management server and their respective instances of the online or mobile application. The trainer can see the user's activities and recovery routines performed. In this way the trainer knows which muscles need attention and can give support to the user remotely (or during periodic in person visits). In some embodiments, a trainer can use the trainer application to put together a workout plan or recovery routine suitable for the user's needs. In some instances, this plan and routine is automatically sent to the user's instance of the online or mobile application (e.g., accessible in a separate tab such as “Trainer Exercises”). In some cases, the trainer application allows the trainer to see which of the suggested recovery exercises were actually performed. In some cases, the trainer and user applications can push notifications between each other.

In various embodiments, the trainer application can also be used or modified for use with a physical therapist. In some instances, the physical therapist application allows a therapist to create a recovery plan for the user. The physical therapist can provide visibility for the therapist into a user's recovery routines and training plans. In this way, the physical therapist can oversee the whole recovery process and intervene to optimize the healing process.

According to various embodiments, features of the trainer application can include: connection of trainer/therapist and user instances of the online or mobile application, creating recovery routines, creating training plans, and/or sending recovery routines.

### Additional Features

In various embodiments, the online or mobile application can feature any combination of the following additional features.

Geolocation: The application can provide targeted content based on user location and time of year. The mobile app experience provides tailored content, ranging from news to customized routines for activities associated with locations. For example, if a user is in Aspen, Colo. during the winter, ski and snowboard routines would be prioritized in the recommendation algorithms and highlighted throughout the app. If a user was at the CrossFit Games, CrossFit content and routines may be highlighted, etc.

Offline storage: The smart recovery devices can store information about usage when not connected to the application. When the smart recovery device connects to the application, the user may get an option to import past sessions. The user can select past sessions to import (all, some, or none). If the application does not have internet service, it also waits for internet connection to pass data to connected services. The smart recovery devices can be configured to not store and share usage data for public settings.

Live recovery: The user can engage in one-on-one or small group chats with a recovery expert(s) via the application. The user can also sign up for live or pre-recorded “recovery classes,” where an expert guides a class through various recovery modalities.

Activity tracker integrations: The application can provide alerts (e.g., haptic) based on specific recovery exercises. For example, the present system can vibrate smart watches to provide cues to users to switch speed, location, or recovery modalities on the smart recovery device. Different vibration patterns can indicate different activities. These haptic cues may guide transitions between body parts, intensity, etc.

Limb mapping: The application can use data associated with compression therapy devices (e.g., NormaTec devices) to provide limb mapping of the user. For example, the application may use calibration data and inflation times to calculate the volume of a limb in attachments by zone. The application can take baseline limb measurements with guided prompts being provided to the user for positioning and best practices. In some examples, the baseline limb measurements can be taken multiple times. The limb measurements can be taken independently, at the start of each session, or at the end of each session. The application may calculate volume changes before and/or after each session to generate a “pliability” score. The application can identify abnormalities (e.g., excess inflammation or swelling from a sprain) and alert the user. In some examples, the application is configured to monitor and provide reports on the identified abnormalities over time. The application can make enhanced recommendations for other recovery products, routines, and exercises based on the limb mapping and monitoring. In certain examples, the application can be configured to combine the limb mapping with data from time-of-flight cameras, such as those included in modern mobile phones. The application may make enhanced recommendations based on the combined data. In one example, the application can receive an indication of the user's body temperature and can adjust one or more device settings to compensate for temperature fluctuations in the body or device.

Time filtering: The application can provide routine recommendations based on how much time the user has available. For example, the application may receive information corresponding to the user's schedule from a calendar app (e.g., Apple Calendar). The application can provide or recommend routines that do not overlap with other events or meetings on the user's schedule.

Most used products: The application can provides data to the user indicating which products are used most based on how many videos the user watched for a particular product. The application can allow the user to track how many minutes of recovery exercises have been done on a particular part of the body.

Tracking product usage: The application can receive data from a smart recovery device to record how long a user is using the device. The user can view this information along with the certain parameters (e.g., speed, pressure) over various timeframes (e.g., per day, per month etc.). The application may present the data as a graph. In some cases, the present application allows one user to compare statistics to other users (e.g., a user who is a professional athlete).

Pressure sensor: The application can receive and display a recovery device's pressure setting in real-time (e.g., by communicating through the Bluetooth connection with the compressive recovery device). In some cases, the application can also display a recommend pressure setting for use with the compressive recovery device.

Quick tips: The application can present exercise, recovery, health or other tips based on a user's preferences and activities. In some cases, the tips can be presented as “pop up” notifications. The notifications may provide an indication to the user of what the smart recovery device is doing in real time.

Quick sorting: In various embodiments, a user can manually enter a sport, product, exercises active per week, recovery body maintenance, or warm up exercise routine. In some cases, the present application filters possible recovery routines relevant to those parameters and organizes the routines based on effectiveness. The application can sort by most popular routines, and routines with highest rating, and based on wearable data. The application can also provide specific routines based on the user's pre-populated selections, sport activity, etc.

Automatic pressure control: The application provides routine sequences based on wearable data activity, sleep, heart rate, etc. This includes variability around time duration, pressure, and zone boost (e.g., increasing time or pressure on a particular zone) of routines being performed with a compression therapy device. At the launch of the routines, the application may send the user a video explaining why the present system selected this routine and what it will do to the user's body. The routines may also be developed by third parties, such as professional athletes who may be in the videos explaining the benefits of the routine. The application can change the pressure in each zone dynamically during the session without user intervention. The pressure may also be adjusted automatically using data (real-time, near real-time, or stored) throughout a routine based on wearable metrics.

Remote control: The application can provide remote control and adjustment of smart recovery devices, such as the compressive recovery devices described herein. For example, the application may enable the user to adjust the massage pattern, session time, time in zone, pressure level, number of pulses, number of zones, boost, and rest time of the recovery devices. In some examples, the adjusted settings can be saved as favorites by the user. As such, the application can save the exact routine configuration for each smart recovery device.

Manual Mode: The application can accept manually input information from the user regarding the activities performed by the user (e.g., 5 mile run, 20 min. swim). Based upon the user's manually input information, the application can provide routine sequences for recovery routines.

Multiple sequences: The application can automatically implement multiple sequences/routines in a series based on data metrics. For example, the application may provide (or control) a series of recovery routines to be performed consecutively based on the analysis of data from wearable devices (e.g., Apple Health, Fitbit, etc.). In some examples, the series of routines can include the use of multiple smart recovery devices, such as the percussive massage applicator and the compression therapy device. For example, the sequence may be to use a percussive recovery device for 5 minutes instructing the user with a video, and then use a compressive recovery device for 30 minutes. The present system may also focus on a particular part of the body during the series of routines. For example, each routine or exercise in the sequence may correspond to a particular part of the body. In some examples, each chamber in the smart recovery device (e.g., leg sleeve of a compression therapy device) can have individual pressure and time control. In certain examples, the series of routines may correspond to different controls for each chamber/zone of the compression therapy device. Custom routines (and sequences) using multiple products (and specific settings and modes for each product) can be saved, such that the user can perform the same custom routine with multiple products at a later date or time

Battery Life indicator: In various embodiments, the online or mobile application shows the battery charge level (or health) of the smart recovery device. The application can also show the number of routines a user can perform based on the devices the user owns and the current battery charge level of each device. For example, the application can inform the user that they have two 30 minute NormaTec recovery sessions left or 40 minutes of Hypervolt Plus recovery time.

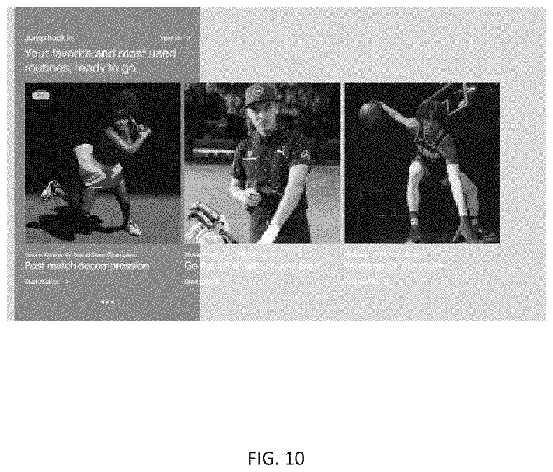

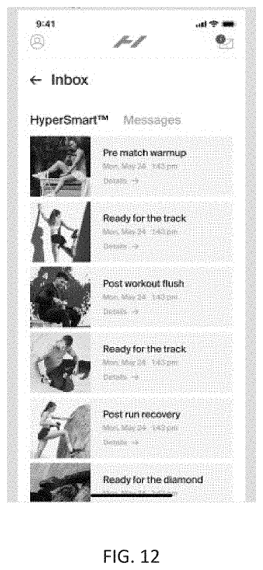

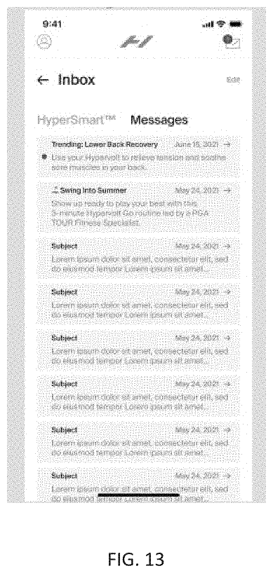

Comparative routines: in various embodiments, a user can select routines recommended or endorsed by elite athletes, practitioners, or sponsored partners (e.g., Equinox™, Soul Cycle™, etc.). The user can also track live (or is notified) when friends, athletes or science advisors are using the present smart recovery device and what specific routine. In one example, users can select a live routine in real time. For example, the application may push a notification to the user that Naomi is “live” and then the user would mimic her specific routine. In certain examples, the athlete or celebrity could opt in or out on whether they want to share a summary profile of the wearable data that prompted the routine. In some cases, the application can track the effectiveness of such routines for the particular user. If a particular routine has high effectiveness for a particular user, the application may present such exercises with more favorability or frequency than other exercises (see FIG. 10). Effectiveness may be measured through the application, e.g., using actual performance data or through user-solicited feedback (e.g., that asks the user how effective a routine was on a scale of 1 to 10). The user can determine where to share this data profile with friends and networks. In addition, the present mobile app allows the athlete to open a text forum or chat where users can send the athlete/celebrity messages and the athlete/celebrity may respond. In some examples, the application provides a messaging feature to facilitate communication between users, athletes, celebrities, and trainers (see FIGS. 12 and 13). In certain examples, the application can provide recommended routines and exercises to the user via the messaging feature.

Sharing routines: The application can enable the user to share routines and/or usage data with their friends via social media platforms (e.g., Twitter, Facebook, etc.). The sharing of routines allows users to see what their friends are doing live and to automatically cross-promote routines and smart recovery devices on social media platforms. In some examples, the user may use the application to recommend specific routines or smart recovery devices to one or more friends.

Weekly prescriptions: in various embodiments, the application offers the user the opportunity to select a multi-week or multi-day prescription guide. The multi-day/week program (e.g., based on a particular injury, a particular goal, a particular sport, etc.) uses the application to deliver a series of routines over a period of time until the user completes the program. For example, the user could select a half-marathon recovery program, and the application would create a recovery series optimized for the devices owned by the user. Likewise, a user through the application may select a four week ankle sprain program. Users may also select a specific multi-week or multi-day program from a specific science advisor, therapist, or elite athlete with recommendations on devices, time-of-day scheduling, and guided recovery. In some cases, a user can connect directly with an athlete or advisor by a chat service and/or telehealth service integrated into the application. In other examples, users can create their own, custom recovery series. The application can integrate into the mobile device's calendar (or a dedicated calendar on the application) to record and show what was done to complete a prescription.

Lifestyle recommendations: The application can provide recommendations to the user based on activity, sleep, heart rate, etc. and other lifestyle descriptors are determined by the user's preselected preferences. For example, the user may indicate that they enjoy using a particular recovery device in the morning after activity.

Sleep score: In various embodiments, the application can receive sleep data from a third party source, e.g., fitness trackers (e.g., Fitbit™, Apple Health™, Whoop™, etc.). The application can then use the data to provide a sleep improvement indicator meter. In various embodiments, a user can select sleep improvement routines and, in some cases, rank them after completing the sleep improvement routines. The application can pull sleep data in comparison to the routines performed with the smart recovery devices to maintain a sleep log and provide a sleep score. In some embodiments, the application can compare actual sleep data to the sleep improvement routines to determine what routine was effective for good sleep for a particular user.

Authenticity scanning: In various embodiments, the application can enable a user to scan a product and determine if the product is authentic (e.g., the scan may be a QR code on a recovery device, or a wireless authentication routine using the Bluetooth connection with a smart recovery device).

Motion detection: In various embodiments, application can receive motion detection information (e.g., through an accelerometer within a smart recovery device) to see how the the product is being used (e.g., angle of use) and provide guidance. The smart recovery device may provide the application with additional data (e.g., temperature changes data) and proximity detection (e.g., send push alert to the application when the device moves).

Proximity detection and security: In various embodiments, the application can track and present to a user a smart recovery product's location. In some examples, the application can provide the ability to lock a stolen device. The application communicates with the backend server if a specific device connects to the server to report its location. The server can communicate with the device to disable it because it has been stolen.

Snooze button: In various embodiments, after the application receives data indicating that a user has completed a workout, application can provide a notification prompting the user to perform a recovery exercise. The user can selects how long the application should wait before sending a recovery exercise routine is provided (e.g., immediately, 5 minutes, 30 minutes, 1 hour, etc.).

Goal tracking: Using the application, a user can set goals for recovery amount per week/day/month, for recovery indexes, or metrics third parties like Apple Health or Garmin (HRV, etc). The application can then track, report, and remind the user if they are behind, and congratulates the user if he/she are ahead on their goals

User Score: in various embodiments, the application can receive data from a user's activity tracking devices (e.g., steps, workouts, sleep, heart rate, etc.) at various intervals (e.g., multiple times per day), and provides a score based on what recovery, maintenance and warm up routines have been completed over a certain time period (e.g., per day, per week, per month), in comparison to the activity, sleep, heart rate, etc. that has been tracked. The user score can also be designated by what activity the activity tracking devices have determined to be completed and how long the activity was performed. In some instances, the application determines how many and what type of recovery, warm up, and maintenance routines a user should perform in connection to how long, and with what devices, the user should use based on the data obtained from the activity tracker devices. In some cases, the application can determines and present the user score in any desirable format; for example, either in a percentage or as a certain category or rating (beginner, average, expert, pro) format. For example, if a user walked 5,000 steps in a day and did a 5 mile run, and had 5 hours of sleep the night before, and the application compares it against what expert information (or in some cases information determined via an artificial intelligence algorithm) deem should have been done. If the user did nothing that day, the user score would be low. If user completed two warm up routines and two recovery routines with a smart recovery device that day, the user score would be higher. In some cases, the user scores may be provided and categorized in subsections for warm up, recovery, and body maintenance scores. The application can measure the duration of time of the recovery warm up or body maintenance routine completed by a user in comparison to the activity levels from a user's activity trackers. For example, if the user worked out with a “Run” for 30 minutes, the application can determine that the user would need to do a certain amount of recovery body maintenance routines for a particular period of time. If the user's run was 60 minutes (double the minutes) the recovery routine may be different. The present system may use artificial intelligence and machine learning to determine what a user normally does, and what the user should do in comparison to others in the user's demographic or in comparison to professional athletes. In such embodiments, the user score can designate various levels beginner, intermediate, basic, advanced professional or derivatives of such based on the user's score. In various embodiments, the application can determine user streaks and awards badges to reward users who achieve certain accomplishment (e.g., achieve a certain ranking for a certain period of time). For example, the application can present, store, and track recovery “status badges” earned by the user by logging the user's recovery activities. In some examples, the application can track usage, total time per device, consistency, etc. to award the status badges. For example, if a user increases their recovery index by 100 points for 12 weeks in a row they become “Silver” status. The present system allows for custom statuses for each product included in the application as well as general statuses.

Diagnostic mode: The application can provide various diagnostic functions. In one example, the application can provide tips when something goes wrong with a smart recovery device and can instruct the user that an issue was detected. For example, the application may detect leaks in the boot or hoses of a compressive recovery device and can provide diagnostic maintenance routines for fixing or repairing the device. Likewise, the application can notify the user of low device battery levels or of connection issues with devices. The application can provide recommendations for user actions or may connect the user with customer service. In addition, the application may send real time device data to customer service (e.g., serial numbers, lot numbers, diagnostic metrics, etc.). In some examples, the application is configured to provide an early warning to the user (or customer service) when one or more parts are near the end of their service life.

Facility/trainer mode: In various embodiments, the application can include a facility/trainer mode configured to manage a plurality of smart recovery devices. For example, the application may monitor in real time the status and function of all devices in a gym or training facility. The application can monitor the service hours of the devices, the locations of the devices (e.g., with low power Bluetooth), and the device inventory of the facility. In some examples, the application can operate in a facility diagnostic mode to monitor the usage of the devices, upcoming service procedures, and the lifespan of parts included in the devices.

Attachment/accessory recommendations: The application can recommend attachments (e.g., different percussive heads) and accessories to be used with the smart recovery devices based on the routines selected from the connected health data.

### Computer-Based Implementations

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

As various changes could be made in the above constructions without departing from the scope of the invention, it is intended that all the matter contained in the above description or shown in the accompanying drawings shall be interpreted as illustrative and not in a limiting sense.

What is claimed is:

1. A method for operating a compressive recovery device, the method comprising:
receiving activity data of a user;
providing a recommended recovery activity based on the activity data, wherein a primary goal of the recovery activity is to facilitate physiological recovery of the user; and
controlling a compressive recovery device to perform the recovery activity on the user.

2. The method of claim 1, wherein the activity data is received from a wearable activity tracker.

3. The method of claim 1, wherein the activity data is received from a smart fitness machine.

4. The method of claim 1, wherein the step of controlling the compressive recovery device comprises controlling a pressure provided by the compressive recovery device.

5. The method of claim 4, wherein controlling the pressure provided by the compressive recovery device to perform the recovery activity includes controlling the pressure in at least one zone of a plurality of zones of the compressive recovery device.

6. The method of claim 1, wherein providing the recommend recovery activity based on the activity data includes providing a sequence of two or more recovery activities, at least one activity of the sequence including the compressive recovery device.

7. The method of claim 1, further comprising the steps of:
connecting to the compressive recovery device; and
receiving recovery data related to the recovery activity from the compressive recovery device.

8. The method of claim 7, further comprising the steps of:
comparing the recovery data to an optimal recovery data; and
presenting the comparison results to the user.

9. The method of claim 8, further comprising the step of presenting the comparison results to a third party.

10. The method of claim 9, wherein the third party comprises at least one of a trainer or a healthcare professional.

11. The method of claim 1, wherein the step of providing a recommended recovery activity comprises using an artificial intelligence technique.

12. A system comprising:
one or more data processing apparatus programmed to perform operations comprising:
receiving activity data of a user;
providing a recommended recovery activity based on the activity data, wherein a primary goal of the recovery activity is to facilitate physiological recovery of the user; and
controlling a compressive recovery device to perform the recovery activity on the user.

13. The system of claim 12, wherein the activity data is received from a wearable activity tracker.

14. The system of claim 12, wherein the activity data is received from a smart fitness machine.

15. The system of claim 12, wherein the operation of controlling the compressive recovery device comprises controlling a pressure provided by the compressive recovery device.

16. The system of claim 15, wherein controlling the pressure provided by the compressive recovery device to perform the recovery activity includes controlling the pressure in at least one zone of a plurality of zones of the compressive recovery device.

17. The system of claim 12, wherein providing the recommend recovery activity based on the activity data includes providing a sequence of two or more recovery activities, at least one activity of the sequence including the compressive recovery device.

18. The system of claim 12, wherein the operations further comprise:
connecting to the compressive recovery device; and
receiving recovery data related to the recovery activity from the compressive recovery device.

19. The system of claim 18, wherein the operations further comprise:
comparing the recovery data to an optimal recovery data; and
presenting the comparison results to the user.

20. The system of claim 12, wherein the operation of providing a recommended recovery activity comprises using an artificial intelligence technique.
